(19) United States
(12) Patent Application Publication
(10) Pub. No.: US 2003/0171258 A1
(43) Pub. Date: Sep. 11, 2003

(54) Particle based vaccine composition

(76) Inventors: Hazire Oya Alpar, London (GB); Ethel Diane Williamson, Salisbury Wiltshire (GB); Leslie William James Baillie, Salisbury Wiltshire (GB)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/335,906

(22) Filed: Jan. 2, 2003

Related U.S. Application Data
(63) Continuation of application No. 09/937,065, filed on Sep. 20, 2001, now abandoned.
Application No. 09/937,065 is a 371 of international application No. PCT/GB00/01108, filed on Mar. 23, 2000.

(30) Foreign Application Priority Data
Mar. 24, 1999 (GB) 9906695.3

(51) Int. Cl. A01N 37/18; A61K 38/00; A61K 9/16; A61K 9/50

(52) U.S. Cl. 514/2

(57) ABSTRACT

A pharmaceutical composition which comprises microparticles comprising (i) a biologically active compound capable of generating an immune response in an animal to which it is administered which is protective against a pathogen; (ii) a polymeric material capable of forming microspheres; and (iii) an immunostimulant comprising a phospholipid. The composition is particularly useful for the oral administration of vaccines.

The present invention relates to compounds for use as immunostimulants, as well as to a composition which is useful for delivering medicaments and particularly vaccines, in particular to mucosal surfaces, for example in oral formulations. The invention further comprises methods of treating individuals using the composition and methods of preparing the composition.

A prime objective in the field of vaccination is the development of a non-parenteral immunisation regimen, which facilitate induction of comparable levels of systemic immunity to that elicited by conventional sub-cutaneous and intra-muscular injections.

The nasapharyngeal passages and pulmonary regions of the respiratory tract represent potential targets for the systemic delivery of peptidergic drugs and vaccines. The relative ease with which therapeutic agents can be inhaled, or introduced into the nose, make these modes of immunisation attractive in terms of probable patient compliance. Furthermore, respiratory mucosae offer certain morphological, physiological and immunological advantages over other non-parenteral sites in terms of immunisation, particularly against pathogenic entitities which affect or utilise mucosal surfaces as portals of entry. This is because effective vaccination against these pathogens normally requires mucosae to the adequately protected with locally produced antibodies of the secretory IgA (sIgA) isotype. Whilst mucosal surfaces are usually poorly protected with IgA following parenteral administration of vaccines, it is now apparent that successful delivery of antigenic material to immunoresponsive elements in mucosa-associated lymphoid tissue (MALT) can result in vigorous stimulation of the mucosal arm of the immune system. By means of the common mucosal immune system (CMIS) it is feasible that several anatomically disparate mucosal surfaces could be protected through mucosal administration of a vaccine at a single site. Mucosal vaccination offers the added advantage that some degree of systemic immunity can be induced in concert with local responses due to translocation of antigenic material from sub-epithelial compartments to systemic immunoresponsive tissues such as the spleen.

Despite the logistical and immunological factors which favour non-parenteral immunisation, simple mucosal application of antigenic proteins, for example in the gastrointestinal or respiratory tracts, is usually ineffectual in terms of vaccination. Enzymatic or chemical destruction, combined with poor absorption into sub-epithelial compartments dictate that mucosally administered vaccines usually require some form of adjuvant or delivery vehicle. One approach is to encapsulate antigenic material within microparticulate polymeric carriers, such as poly-DL-lactide (PLA) microspheres (Vaccine 1994, 12, 5-11). Such procedures serve to protect labile vaccines from lumenal degradation and enhance absorption into mucosal and systemic compartments (J. H. Eldridge et al., Seminars in Hematology, (1993), 30, 16-25). There is good evidence that microencapsulation may also adjuvantise by converting soluble antigenic molecules into particulate species, thus promoting vaccine uptake into antigen presenting cells (APC)(Y. Tabata et al., Adv. Polym. Sci. (1990), 94, 107-141, L. Vidard et al., J. Immunol. (1996), 156, 2809-2818, N. Van Rooijen, Immunol. Today (1990) 11, 436-439) or microfold cells (M-cells) in lymphoid follicles (R. I. Walker et al., Vaccine, 12, 387, 1994, D. T. O'Hagan et al,, Vaccine, 1989, 7, 421-424, P. G. Jenkins et al., J. Drug Targetting, 1995, 3, 79-81). Nasal delivery of microsphere formulation of vaccine has also been described (A. J. Almeida et al., J. Pharm & Pharmacology, 25, 198-203 1993, H. O. Alpar et al., J. Drug Targeting 2/2, 147-149, 1994, A. J. Almeida et al., J. Drug Targeting 3(b), 255-467 1996).

WO 91/06282 describes certain drug compositions which are suitable for intranasal delivery and which include absorption enhancers. Examples of suitable enhancers are lysophosphtidylcholine.

The applicants have found that the presence of a phospholipid in a vaccine formulation has an immunostimulant effect, over and above that which could be explained by absorption enhancer effects.

Thus the invention provides a phospholipid for use as an immunostimulant.

As used herein, the term “immunostimulant” refers to an adjuvant which stimulates the immune system of a host animal to which it is administered and thereby increases the protective effect produced by a protective antigen administered to that animal, as compared to the effect which would be produced by administration of the protective antigen alone.

In particular, it has been noted that a microencapsulated biologically active formulation, especially of immunogens, which comprises a phospholipid in addition to the polymeric material used in the formation of the microparticles, has an increased biological effect. This may be particularly noticed when the formulation is administered by any route including parenteral or other routes, but is particularly effectively when administered by way of a mucosal surface.

Thus the invention provides a pharmaceutical composition, which comprises microparticles comprising

(i) biologically active agent which is capable of generating an immune response in an animal to which it is administered;

(ii) a polymeric material capable of forming microspheres; and

(iii) an immunostimulant comprising a phospholipid.

In one embodiment, the composition is suitable for parenteral administration. A particular example is intramuscular (i.m.) administration.

In a preferred embodiment, the composition is suitable for non-parenteral administration for example to mucosal surfaces.

Administration to mucosal surfaces may be effected by oral application, by pulmonary application, for example by intra-tracheal administration, or particularly by intra-nasal application. In particular, the compositions of the invention are administered by the oral route.

The polymeric material used in the compositions of the invention is suitable for forming microparticles (sometimes known as microcapsules or microspheres). It may be a low, medium or high molecular weight polymer. Examples of low molecular weight polymers are polymers which have a molecular weight of between 0.1 and 10 kDa, more preferably between 1 and 5 kDa and typically about 2-3 kDA.

The use of high molecular weight polymers in the encapsulation of a tetanus vaccine for intramuscular administration has been described (Vaccine 1994, 12, 4, 299-306). A formulation of microencapsulated ricin toxoid vaccine which is applied intranasally has also been described (Vaccine 1994, 14, 11 1031). However, in that case, high molecular weight polymer microparticles (94 kDa) were less effective than those prepared from a copolymer of lower molecular weight (72 kDa).

The polymeric material used in the composition of the present invention suitably has a high molecular weight in excess of 94 kDa, for example of 100 kDa or more.

A particularly suitable polymeric material for use in the compositions of the invention comprises a poly(hydroxy)acid or or a copolymer thereof. A particular example is poly-(L-lactide) or PLA but other high molecular weight polymeric material such as poly(lactic/glycolic acid), polycyonacrylates, polyanhydrides, polycarbonates or polycaprolactones as are known in the art may be employed.

Examples of suitable phospholipids for use in the microparticles of the compositions of the invention include many pharmaceutically acceptable phospholipids or precursors therefore, which may be cationic, anionic or neutral in character. These include lecithin or its precursor phosphoryl choline, distearylphosphatidylcholine (DSPC) and phosphatidinylserine (PS). Examples of positively charged lipids include dipalmitoylphosphatidylcholine (DPPC) and dioleoyltrimethylammoniumpropane (DOTAP). A particularly preferred phospholipid is lecithin which is widely available and commonly used in other types of pharmaceutical composition.

Suitably the phospholipid is added to the composition in an amount of from 0.1% to 20% w/w, and preferably about 5% w/w.

The microparticles may optionally further comprise agents which stabilise emulsions such as polyvinylalcohol, methyl cellulose or dextrans.

They will suitably be of an average size of from 0.1 μm to 10 μm in diameter.

These compositions may be used to deliver a biologically active agents which are capable of generating a protective immune response in an animal, particularly a mammal, to which it is administered. Examples of such agents include antigenic polypeptides as well as nucleic acid sequences which may encode these polypeptides and which are known as “naked DNA” vaccines.

As used herein the expression “polypeptide” encompasses proteins or epitopic fragments thereof.

Suitable polypeptides are sub-unit vaccines or others such as tetanus toxoid, diptheria toxoid and Bacillus anthracis protective antigen (PA).

In one embodiment, the composition of the invention comprises a biologically active agent which is capable of generating a protective immune response against Yersinia pestis. The agent is suitably a sub-unit vaccine, for example as described in WO 96/28551. The vaccine described and claimed there comprises a combination of the V antigen of Y. pestis or an immunologically active fragment thereof or a variant of these, and the F1 antigen of Y. pestis or an immunologically active fragment thereof or a variant of these.

As used herein, the term “fragment” refers to a portion of the basic sequence which includes at least one antigenic determinant. These may be deletion mutants. One or more epitopic region of the sequence may be joined together.

The expression “variant” refers to sequences of nucleic acids which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% homologous, preferably at least 75% homologous, and more preferably at least 90% homologous to the base sequence. Homology in this instance can be judged for example using the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

Preferably, vaccine compositions will further comprise a conventional adjuvant in order to increase or enhance the immune response to the biologically active material administered. Suitable adjuvants include pharmaceutically acceptable adjuvants such as Freund's incomplete adjuvant, alhydrogel, aluminium compounds and, preferably adjuvants which are known to up-regulate mucosal responses such as CTB, the non-toxic pentameric B subunit of cholera toxin (CT).

They may also comprise other known composition components such as colouring agents and preservatives and in particular cetrimide. These are suitably present in amounts of from 0.1 to 0.7% w/v.

In a particular embodiment, the microspheres used in the compositions may further comprise an S-layer proteins, in particular, S-layer proteins derived from a bacteria against which the biologically active agent produces a protective immune response. These proteins are suitably coated onto the surface of the particles. It has been shown (Sleyr et al., Crystalline bacterial cell surface proteins. Biotechnology Intelligence Unit, 1996, R. G. Landes Company and Academic Press Inc.) that the stability of liposomes can be increased by such coatings. S-layer proteins are found on the surface of most bacteria and form a regular two dimensional array known as an S-layer. Isolated S-layer proteins are able to form entropy driven monomolecular arrays in suspension, and on the surface of structures such as liposomes.

Compositions of the invention may be suitable for oral and intranasal application. They are particularly effective when applied orally.

They may comprise microparticles per se which are optionally preserved, for example by lyophilisation, or the microparticles may be combined with a pharmaceutically acceptable carrier or excipient. Examples of suitable carriers include solid or liquid carriers as is understood in the art.

The invention further provides a method of producing a pharmaceutical composition, which method comprises encapsulating a biologically active agent as described above in a polymeric material which suitably has a high molecular weight and in particular a molecular weight of 100 kDa or more, in the presence of a phospholipid such as lecithin. The phospholipid may be incorporated within the microparticle, or at the surface, of preferably is distributed throughout the microparticle.

Methods of forming liposomes are well known in the art. They include dispersion of dehydrated lipid films into an aqueous media, emulsion techniques and lyophilisation methods as are well known in the art.

Microparticles of the invention are suitably prepared using a double emulsion solvent evaporation method. Briefly, the biologically active agent, in solution or in a suitably lyophilised state, is suspended or dissolved in an aqueous solution of the polymeric material such as polyvinyl alcohol (PVA) and the phospholipid such as lecithin. A solution of the polymer in an organic solvent such as dichloromethane, is added with vigorous mixing. The resultant emulsion is then dropped into a secondary aqueous phase, also containing polymeric (PVA or the like) and optionally also the phospholipid with vigorous stirring. After addition, the organic solvent is allowed to evaporate off and the resultant microspheres separated.

The compositions of the invention will suitably comprise an appropriate dosage unit of the active agent. This will vary depending upon the nature of the active agent being employed, the nature of the patient, the condition being treated and other clinical factors. In general however, the composition of the invention will comprise approximately 0.1 to 10 wt % of active ingredient.

The amount of polymer in the composition will be of the order of 70 to 99 wt % of the composition, and suitably from 90 to 99 wt % of the microparticle components will be the polymeric material. The amount of phospholipid, will be of the order of 0.1 to 10 wt % of the composition.

In use, a reasonable dosage for nasal administration would be of from 0.05 g to 0.2 g. A formulation for oral administration to humans will generally contain, for example, from 0.5 mg to 2 g of active agent within a composition as defined above.

Preferred compositions of the inventions are vaccine compositions where the biologically active agent is able to produce a protective immune response to a pathogenic organism as described above. Thus, in a further aspect, the invention provides a method of protecting a mammal against infection, which method comprises administration of a vaccine composition as described above to mammal. In particular, the composition is applied to a mucosal surface, in particular gastrointestinal surface, of a mammal.

The applicants have shown that through the oral administration of tetanus toxoid in accordance with the present invention, antibody titres were achieved which are in excess of those associated with protection from the relevant toxin, and protection to a subcutaneous challenge of tetanus toxoid was provided.

A further aspect of the invention comprises the use of a phospholipid as an immunostimulant in the production of a vaccine for use in prophylactic or therapeutic treatment.

The invention will now be particularly described by way of example with reference to the accompanying drawings in which:

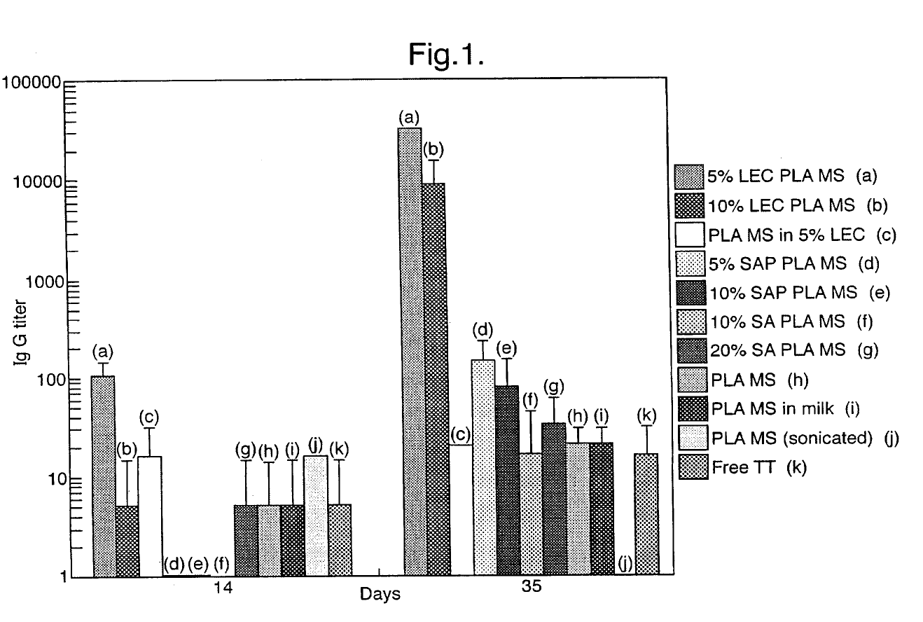

FIG. 1 illustrates the serum immune response in mice to orally delivered microencapsulated and free tetanus toxoid with 50 lf units on day 1, 3, 5 and boosted on day 28, 30 and 50;

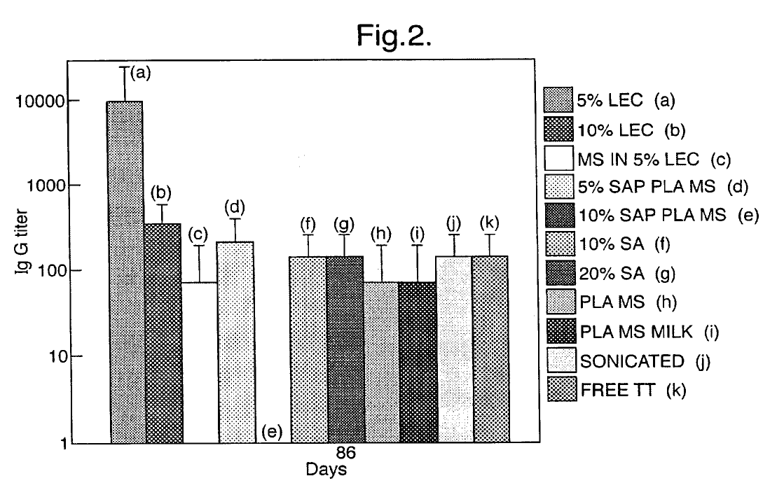

FIG. 2 illustrates the serum immune immune response after 86 days; and

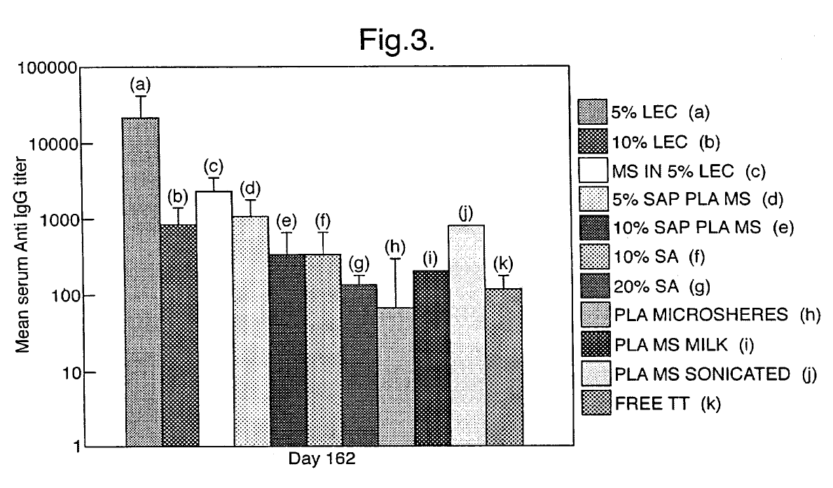

FIG. 3 illustrates the serum immune immune response after 162 days following a subcutaneous “mock challenge” with tetanus toxoid.

### EXAMPLE 1

Microencapsulation of Tetanus Toxoid

Poly-L-lactide of molecular weight 100 kDa (Polysciences Inc. USA) was used in a modification of the double emulsion solvent evaporation method (Y. Ogawa et al., Chem. Pharm. Bull., 36 (1988) 1095-1103). Briefly, 1.5 ml of a 1.5% w/v aqueous solution of polyvinyl alcohol (PVA)(13-25 k) containing tetanus toxoid (4000 Lf units) was prepared. This formed the aqueous phase. An organic phase was prepared separately by mixing with 200 mg of 100 K PLA polymer dissolved in 5 ml of HPLC grade dichloromethane (DCM) with 5% w/w lecithin. The two phases were homogenised together using a Silverson homogeniser (Silverson, UK) for 1 minute. The resultant primary emulsion was added, drop by drop, into a secondary aqueous phase (75 ml) comprising 1.5% w/v PVA and 0.1% w/v lecithin. The mixture was then homogenised using a Silverson homogeniser for 5 minutes. This secondary phase was gently stirred overnight to until the dichloromethane had evaporated. Microspheres were recovered by centrifugation, washed with double distilled water three times and then lyophilised.

Other microspheres using 10% w/w lecithin instead of the 5% w/w solution were prepared using a similar procedure (10% LEC PLA MS). Further microspheres where the lecithin was omitted entirely from the preparation were also prepared by an analgous method but 5% w/w lecithin was used instead of water alone for the oral administration of microspheres (PLA MS in 5% LEC).

In addition, this method was used to prepare comparative microspheres where the lecithin in the primary emulsion was replaced by either 5% w/w or 10% w/w saponin (SAP) or 10% w/w or 20% w/w stearyl amine (SA) and lecithin omitted from the preparation entirely. Finally, PLA microspheres without any adjuvant were prepared.

### EXAMPLE 2

Immunisation Study Groups of three Balb/c female mice were orally dosed with the microspheres prepared in Example 1 containing 50 LF tetanus toxoid on days 1, 3 and 5 of the trial, and boosted on day 28 and 30. Another group of mice was orally dosed with similar amounts of tetanus toxoid but in free solution.

Serum immune responses were monitored. Tail vein blood samples were taken from all animals on days 14 and 35 of the experiment. Titration of IgG antibody isotypes in serum samples was achieved using an ELISA. Briefly, individual serum samples were aliquoted to microtitre plates pre-coated with tetanus toxoid. Binding of serum antibody was detected with peroxidase-labelled secondary antibody to mouse IgG (Sigma A4416). Antibody titre was estimated as the maximum dilution of the serum giving an absorbance reading greater that the maximum optical density (OD) of titrated naïve serum. From this, mean titres ± standard deviation (SD) were derived per treatment group.

At day 162, the immune responses following a subcutaneous “mock challenge” with TT as monitored.

The results are shown in FIGS. 1-3. In these Figures, the following letters represent compositions prepared as described above as follows:

(a) 5% lecithin containing PLA microsphere composition:

(b) 10% lecithin containing PLA microsphere composition;

(c) PLA microspheres in the presence of 5% lecithin;

(d) 5% w/w saponin containing microsphere composition;

(e) 10% w/w saponin (SAP) containing microsphere composition;

(f) 10% w/w stearyl amine (SA) containing microsphere composition;

(g) 20% w/w stearyl amine (SA) containing microsphere composition;

(h) PLA microspheres;

(i) PLA microspheres in milk;

(j) PLA micropheres which have been sonicated;

(k) Free tetanus toxoid.

Microsphere formulations containing lecithin showed an amplification of >3000 as compared to free tetanus toxoid and other microsphere formulations. Microspheres which contained no lipid in a phospholipid vehicle gave similar results to the free tetanus toxoid.

Even after 86 days of the dosing and following the s.c. challenge at day 162, titres were above the protective titre levels (FIGS. 2 and 3) when formulations in accordance with the invention were employed.

1. A pharmaceutical composition which comprises microparticles comprising
(i) a biologically active compound capable of generating an immune response;
(ii) a polymeric material capable of forming microspheres; and
(iii) an immunostimulant comprising a phospholipid.

2. A composition according to claim 1 which is adapted for administration to a mucosal surface.

3. A composition according to claim 2 which is adapted for oral administration.

4. A composition according to any one of the preceding claims wherein the polymeric material is a high molecular weight polymer.

5. A composition according to claim 4 wherein the polymeric material has a molecular weight of 100 kDa or more.

6. A composition according to any one of the preceding claims wherein the polymeric material comprises a poly(hydroxy)acid or or a copolymer thereof.

7. A composition according to any one of the preceding claims wherein the phospholipid is selected from lecithin or its precursor phosphoryl choline, distearylphosphatidylcholine (DSPC) and phosphatidinylserine (PS).

8. A composition according to claim 7 wherein the phospholipid is lecithin.

9. A composition according to any one of the preceding claims which contains from 0.1% to 20% w/w phospholipid.

10. A composition according to claim 9 wherein the amount of phospholipid is about 5% w/w.

11. A composition according to any one of the preceding claims wherein the biologically active compound capable of generating an immune response in an animal to which it is administered comprises a polypeptide or a nucleic acid.

12. A composition according to claim 11 wherein the biologically active agent comprises tetanus toxoid, diptheria toxoid, Bacillus anthracis protective antigen (PA) or an agent which is capable of generating a protective immune response against Yersinia pestis.

13. A composition according to claim 11 or claim 12 which further comprises a conventional adjuvant in order to increase the immune response to the biologically active material administered.

14. A composition according to any one of the preceding claims which further comprises a preservative.

15. A composition according to claim 14 wherein the preservative is cetrimide and this is present in amounts of from 0.1 to 0.7% w/v.

16. A composition according to any one of the preceding claims wherein the microspheres used in the compositions may further comprise an S-layer proteins.

17. A composition according to claim 16 wherein the biologically active agent is as defined in claim 10 and wherein the S-layer proteins are derived from a bacteria against which the biologically active agent produces a protective immune response.

18. A composition according to any one of the preceding claims wherein the microspheres are suspended in a diluent or carrier.

19. A composition according to any one of the preceding claims wherein the ratio of the polymeric material to the phospholipid is from 99:1 to 9:1% w/w.

20. A method of producing a pharmaceutical composition according to claim 1, which method comprises encapsulating a biologically active agent which is capable of generating a protective immune response, in a polymeric material, in the presence of a phospholipid.

21. A method according to claim 20 wherein the phospholipid is distributed throughout the microparticle.

22. A method according to claim 20 or claim 21 wherein the composition comprises a prophylactic or therapeutic vaccine.

23. A method of protecting a mammal against infection by a pathogen, which method comprises administration of a composition according to any one of claims 1 to 19 wherein the biologically active material is able to generate a protective immune response against said pathogen, to a mammal.

24. A method according to claim 23 wherein the composition is applied to a mucosal surface of said mammal.

25. A method according to claim 24 wherein the mucosal surface comprises a gastrointestinal surface.

26. The use of a phospholipid in the production of a microsphere for use in the composition according to claim 1.

27. A phospholipid for use as an immunostimulant.

28. The use of a phospholipid as an immunostimulant in the production of a vaccine for use in prophylactic or therapeutic treatment.
